(19) United States
(12) Patent Application Publication
(10) Pub. No.: US 2007/0082950 A1
(43) Pub. Date: Apr. 12, 2007

(54) Method for producing vitamin a acetate

(75) Inventors: Kai Michael Exner, Eppelheim (DE); Klemens Massonne, Bad Durkheim (DE); Harald Laas, Maxdorf (DE); Detlev Glas, Frankenthal (DE); Laszlo Szarvas, Ludwigshafen (DE)

Correspondence Address: CONNOLLY BOVE LODGE & HUTZ, LLP, P O BOX 2207, WILMINGTON, DE, 19899, US

(73) Assignee: BASF AKTIENGESELLSCHAFT, LUDWIGSHAFEN (DE)

(21) Appl. No.: 10/580,958

(22) PCT Filed: Dec. 14, 2004
(86) PCT No.: PCT/EP04/14209

§ 371 Date: May 31, 2006

(30) Foreign Application Priority Data
Dec. 17, 2003 (DE) 10359433.7

(51) Int. Cl. C07C 67/02; A61K 31/22

(52) U.S. Cl. 514/546; 560/260

(57) ABSTRACT

The present invention relates to a process for preparing vitamin A acetate by reacting β-vinylionol with triphenylphosphine in the presence of sulfuric acid in a solvent mixture consisting of 60 to 80% by weight methanol, 10 to 20% by weight water and 10 to 20% by weight aliphatic, cyclic or aromatic hydrocarbons having 5 to 8 carbon atoms to give β-ionylideneethyltriphenylphosphonium salts and subsequent Wittig reaction with 4-acetoxy-2-methylbut-2-enal.

The present invention relates to a process for preparing vitamin A acetate (VAA) by reacting β-vinylionol with triphenylphosphine in the presence of sulfuric acid to give β-ionylideneethyltriphenylphosphonium salts (C15 salt) followed by Wittig reaction with 4-acetoxy-2-methylbut-2-enal (C5 acetate).

Vitamin A acetate is an important industrial product which is widely used in the pharmaceutical and cosmetic sectors and in food products and food supplements and as feed additive in animal nutrition.

DE-A 2729974 describes an industrial synthesis of C15 salt starting from β-vinylionol by reaction with triphenylphosphine in the presence of sulfuric acid. Lower aliphatic alcohols, especially methanol, are described as solvents.

Curley et al. describe in J. Org. Chem. 1984, 49, 1941-44 the same reaction in methanolic solution in the presence of HBr.

DE-A 1279677 discloses a continuous process for carrying out the Wittig reaction of C15 salt with C5 acetate in methanolic solution at temperatures below 5° C.

Management of the reaction in two-phase systems composed of water and halogenated organic solvents at temperatures of from 0 to 60° C. is described in DE-A 2636879.

DE-A 2733231 describes an embodiment of the Wittig reaction of various C15 salts with C5 acetate in water at temperatures of from 0 to about 100° C. Ammonia is disclosed as base, besides alkali metal carbonates. Reaction of the C15 salts obtained by using sulfuric acid, a hydrogen sulfate or phosphoric acid takes place particularly expediently at room temperature.

In view of the industrial complexity of vitamin A acetate syntheses, there is still a need to optimize and thus make more economic the individual stages in the overall process and thus the complete preparation process.

It is an object of the present invention to provide a process which permits conversion of β-vinylionol into vitamin A acetate to be carried out in an industrially and economically advantageous temperature range with high conversion and high space-time yield.

We have found that this object is achieved by providing a process for preparing vitamin A acetate of the formula (I) 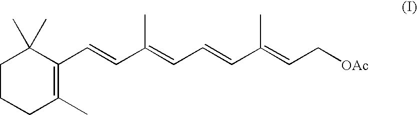
by reacting β-vinylionol of the formula (II) 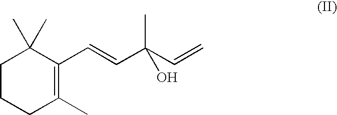
with triphenylphosphine in the presence of sulfuric acid to give the C15 salt of the formula (III) 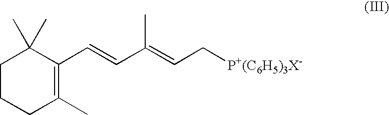
where X⁻ is HSO₄⁻ and/or CH₃SO₄⁻, and subsequent Wittig reaction with C5 acetate of the formula (IV) 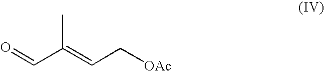
in water as solvent and in the presence of a base, wherein the synthesis of C15 salt of the formula III starts from β-vinylionol at a temperature of from 45 to 55° C. in a solvent mixture consisting of
60 to 80% by weight methanol,
10 to 20% by weight water and
10 to 20% by weight aliphatic, cyclic or aromatic hydrocarbons having 5 to 8 carbon atoms,
   where the % by weight data chosen within the stated ranges must add up to 100% by weight.

β-Vinylionol prepared in any way is suitable for preparing the C15 salt. The β-vinylionol normally employed has a purity of about 90 to about 99%, preferably a purity of about 90 to about 95%.

All the compounds having one or more olefinic unsaturations which are mentioned for the purposes of the present invention may be employed or obtained in the form of their respective possible double-bond isomers or in the form of mixtures thereof.

Commercially available triphenylphosphine for example is suitable for the conversion of β-vinylionol. The triphenylphosphine employed for the purposes of the process of the invention advantageously has a purity of about 95 to about 99.9%, preferably of about 98 to about 99.9%. The amount of triphenylphosphine employed is, based on β-vinylionol, ordinarily approximately equimolar, preferably approximately 0.95 to approximately 1.05 equivalents. It is often advantageous to employ triphenylphosphine in slightly less than stoichiometric amount based on β-vinylionol, i.e. from approximately 0.95 to approximately 0.995 equivalent.

The dissolving medium used when carrying out the C15 synthesis according to the invention comprises mixtures of methanol and water which additionally also comprise further organic solvents. Aqueous methanol is ordinarily used, with methanol normally being present in excess. A further organic component is also added to the solvent mixture, for example a hydrocarbon having 5 to 8 carbon atoms, which may be aliphatic, cyclic or aromatic, such as, for example, hexane, heptane, octane, isooctane, cyclohexane, toluene, cyclopentane, methylcyclopentane, dimethylcyclopentane (1,1-, 1,2-, 1,3-, 1,4-), ethylcyclopentane, 2-methylhexane, 3-methylhexane, 2-methylheptane, 3-methylheptane, 4-methylheptane, 2-ethylhexane, 3-ethylhexane, methylcyclohexane, dimethylcyclohexanes (1,1-, 1,2-, 1,3-, 1,4-) and more of the like or mixtures thereof. Instead of adding said hydrocarbons, it is also possible to use methanol which already comprises the hydrocarbons as impurity. It has proved to be particularly advantageous to add alkanes such as, for example, heptane, cyclohexane, octane, isooctane or mixtures thereof. It has moreover emerged that the progress of the reaction depends on the composition of the dissolving medium. Good results are usually achieved on use of ternary solvent mixtures consisting of methanol, water and heptane, and the heptane used may also comprise up to about 40% by weight of further hydrocarbons having about 5 to about 8 carbon atoms.

The solvent mixtures preferably employed in the C15 salt preparation of the invention consist of about 64 to 72% by weight methanol, about 14 to 18% by weight water and about 14 to 18% by weight heptane, which may comprise up to 40% by weight of further hydrocarbons. Very particularly preferred solvent mixtures consist of about 66.5% by weight methanol, about 16.5% by weight water and about 17% by weight heptane, it also being possible to use heptane mixed with other hydrocarbons as mentioned above instead of heptane.

The concentration of the reagents in the chosen solvent mixture can in principle be varied over a wide range. However, taking account of the economic aspect, it is advantageous not to use too great a dilution. Concentrations, based on the amount of the complete reaction mixture, of about 16 to about 24% by weight, preferably about 18 to about 22% by weight, β-vinylionol and about 18 to about 26% by weight, preferably about 20 to about 24% by weight, triphenylphosphine have proved expedient.

The solvent mixtures employed are, after completion of the reaction, separated from the reaction products and preferably reused, for example in a further reaction of the invention of β-vinylionol with triphenylphosphine to give the C15 salt. Changes in the composition of the solvent mixture caused thereby can be compensated by adding additional amounts of the respective components. Changes in the composition of the alkane component, for example through an increase or decrease in the concentration of individual hydrocarbons, are not critical as long as they do not have a noticeable unfavorable effect on the progress of the reaction.

Reaction of β-vinylionol with triphenylphosphine to give the C15 salt is carried out according to the invention in the presence of sulfuric acid. The concentration of the sulfuric acid can be varied over a wide range and is ordinarily about 50 to about 96% by weight. The concentration of the sulfuric acid employed is preferably about 60 to about 90% by weight, preferably about 70 to about 80% by weight. The sulfuric acid concentration is very particularly preferably about 73 to about 77% by weight. It is employed in approximately equimolar amount based on the β-vinylionol to be converted, i.e. in an amount of about 0.9 to about 1.1 equivalents. It is advantageous to employ a slight excess of sulfuric acid, i.e. about 1.01 to about 1.1 equivalents.

The C15 salt synthesis of the invention is usually carried out by introducing triphenyl-phosphine into the chosen solvent mixture and adding the required amount of sulfuric acid at temperatures of about 30 to about 50° C. The sulfuric acid is preferably added in portions or continuously over a lengthy period (about 1 to about 10 h). The chosen amount of β-vinylionol is then added, and the temperature is advantageously adjusted to about 45 to about 55° C. The reaction is ordinarily complete after about 2 to about 20 h. The resulting reaction mixture can be worked up in a manner known to the skilled worker.

The C15 salt of the formula III obtained in this way ordinarily results in the form of a mixture consisting of the hydrogen sulfate (X=HSO₄) and the methyl sulfate (X=CH₃SO₄). Preferred reaction products comprise, besides the predominantly formed hydrogen sulfate, as little as possible, for example about 0.1 to about 15 mol %, of the methyl sulfate. Particularly preferred C15 salt, especially for the purposes of the further reaction according to the invention to give vitamin A acetate, comprises only about 0.1 to about 5 mol % of the methyl sulfate.

The resulting C15 salt is converted according to the invention by reaction with the aldehyde of the formula IV (4-acetoxy-2-methylbut-2-en-al), which is referred to as C5 acetate, into vitamin A acetate. The C5 acetate to be employed does not need to satisfy the special requirements. It is ordinarily employed in a purity normally expected for chemical intermediates, i.e. in a purity of about 90 to about 99%. Reaction with the C15 salt obtained according to the invention is carried out in water or aqueous solvent mixtures which may comprise for example, alcohols having 1 to 4 carbon atoms such as, for example, methanol, ethanol, propanol or isopropanol. The reaction is preferably carried out in water.

The Wittig reaction is advantageously carried out by heating a solution or a mixture of the C15 salt in the chosen solvent to about 45 to about 55° C., preferably about 48 to about 52° C., and adding a suitable base such as, for example sodium hydroxide solution, potassium hydroxide solution, alkali metal or alkaline earth metal hydroxides, alkaline earth metal oxides such as, for example MgO or BaO, sodium carbonate, potassium carbonate or other basic carbonates, alcoholates or amines such as, for example, triethylamine or mixtures of said compounds. A base which is preferred for the purposes of the process of the invention is ammonia, which is advantageously employed in an amount, based on the amount of C15 salt to be reacted, of about 2 to about 2.3 equivalents. Ammonia is particularly preferably employed in an amount of from 2.1 to about 2.2 equivalents.

The chosen amount of ammonia can be introduced into the reaction mixture or the reaction solution in various forms. Thus, for example, gaseous or liquid ammonia can be passed into the reaction mixture or deposited in vapor or droplet form on the surface thereof. Ammonia is preferably added in the form of aqueous solutions which may comprise, for example, about 5 to about 20% by weight ammonia. Preferred solutions comprise about 9 to about 15% by weight ammonia.

In parallel with the addition of the base, or else with a time lag relative thereto, C5 acetate is added in a molar amount approximately corresponding to the amount of C15 salt to be reacted, i.e. about 0.9 to about 1.1 equivalents, to the reaction mixture. The reagents are advantageously added in portions or continuously. They are ordinarily metered in over a period of about 1 to about 5 h. The reaction mixture can then be subsequently stirred still in the stated temperature range or, if appropriate, else at lower or higher temperatures. The reaction mixture can be worked up by methods known per se to the skilled worker, for example by extraction.

The process of the invention is suitable for reactions on any scale. It can be carried out batchwise, semicontinuously or completely continuously with good results. The particular efficiency of the process is evident especially in reactions on the industrial scale. In this case, the semicontinuous or completely continuous embodiment of the process steps offers distinct advantages in relation to process technology and in relation to economics. In the continuous or semicontinuous embodiment of the process, all the stated times influenced thereby, such as, for example, reaction times, metering times and the like, are to be understood as average times.

It emerges, especially when the process is carried out semicontinuously or completely continuously, but also when the process of the invention is carried out batchwise, that the stated process parameters often cannot be varied independently of one another.

In one particularly preferred embodiment of the process of the invention, accordingly, 0.98 equivalent of triphenylphosphine is introduced into a solvent mixture consisting of 66.5% by weight methanol, 16.5% by weight water and 17% by weight heptane in a concentration of 32% by weight at 40° C. with stirring, and 1.02 equivalents of approximately 75% by weight sulfuric acid are added dropwise over the course of about 1 h. Then, at about 50° C., 1.0 equivalent of β-vinylionol is added and stirred at about 50° C. until the reaction is complete. Working up and isolation of the C15 salt obtained as reaction product can be carried out in a manner known to the skilled worker.

Following this, preferably 1 equivalent of the C15 salt obtained in this way is heated to a temperature of about 50° C. and, while stirring, 2.1 to 2.2 equivalents of an approximately 12% by weight aqueous ammonia solution and 1.0 to 1.1 equivalents of C5 acetate are metered in. After completion of the reaction, the mixture is worked up and purified in a conventional way.

The following examples serve to illustrate the invention without, however, restricting it in any way:

### EXAMPLE 1

### Preparation of C15 salt

139.7 g of triphenylphosphine were introduced into a solvent mixture consisting of 206.8 g of methanol, 44.46 g of water and 40.68 g of heptane at 40° C. with stirring. Over the course of 1 h, 72.7 g of 75% strength sulfuric acid were added dropwise. Then 130 g of β-vinylionol with a purity of 92.1% were metered in over the course of 2 h, the temperature was raised to 50° C., and the mixture was stirred for 4 h. Extractive workup resulted in C15 salt in a yield of 99.9% (based on triphenylphosphine employed).

### EXAMPLES 2 to 5

### Preparation of vitamin A acetate

A solution of 100 g of C15 salt in 150 g of water was heated to 50° C., and the amount of ammonia indicated in table 1, and 1.0 to 1.1 equivalents of C5 acetate were metered in and, after the addition was complete, the mixture was stirred at the chosen reaction temperature (see table 1) for 30 min. Extractive workup of the reaction mixture resulted in vitamin A acetate in yields of from 82 to 89%.

TABLE 1

| | NH₃ | Reaction temp. | Yield |
|---|---|---|---|
| Example | equiv. | [° C.] | [%] |
| 2 | 2.0 | 50 | 82 |
| 3 | 2.1 | 50 | 89 |
| 4 | 2.2 | 50 | 88 |
| 5 | 2.0-2.2 | 34 | 77-82 |

1. A process for preparing vitamin A acetate of the formula (I) 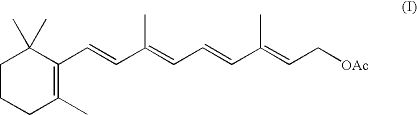
by reacting β-vinylionol of the formula (II) 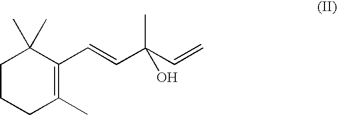
with triphenylphosphine in the presence of sulfuric acid to give the C15 salt of the formula (III) 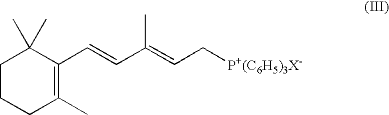
where X⁻ is HSO₄⁻ and/or CH₃SO₄⁻, and subsequent Wittig reaction with C5 acetate of the formula (IV) 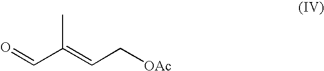
in water as solvent and in the presence of a base, wherein the synthesis of C15 salt of the formula III starts from β-vinylionol in a solvent mixture consisting of
60 to 80% by weight methanol,
10 to 20% by weight water and
10 to 20% by weight aliphatic, cyclic or aromatic hydrocarbons having 5 to 8 carbon atoms,
where the % by weight data chosen within the stated ranges must add up to 100% by weight.

2. The process according to claim 1, wherein the Wittig reaction is carried out at a temperature of from 45 to 55° C. in the presence of, based on the C15 salt employed, from 2 to 2.3 equivalents of ammonia as base.

3. The process according to claim 1, wherein the synthesis of C15 salt of the formula III is carried out at a temperature of from 45 to 55° C.

4. The process according to any of claims 1, wherein the synthesis of C15 salt of the formula III is carried out in the presence of sulfuric acid with a concentration of from 70 to 80% by weight.

5. The process according to any of claims 1, wherein
a. the synthesis of C15 salt of the formula III is carried out at a temperature of from 48 to 52° C. in a solvent mixture consisting of
64 to 72% by weight methanol,
14 to 18% by weight water and
14 to 18% by weight heptane which may comprise up to 40% by weight of further hydrocarbons, and
b. the Wittig reaction is carried out at a temperature of from 48 to 52° C. in the presence of, based on the C15 salt employed, from 2.1 to 2.2 equivalents of ammonia as base.

6. The process according to any of claims 1, wherein the synthesis of C15 salt of the formula III is carried out in the presence of sulfuric acid with a concentration of from 73 to 77% by weight.

7. The process according to any of claims 1, wherein the Wittig reaction is carried out by employing C15 salt of the formula III in the form of a mixture consisting of the hydrogen sulfate (X=HSO₄) and the methyl sulfate (X=CH₃SO₄), where the proportion of methyl sulfate is from 0.1 to 15%.

8. The process according to claim 1, wherein the proportion of methyl sulfate is from 0.1 to 5%.

9. The process according to claim 1, wherein ammonia is employed in the Wittig reaction in the form of an aqueous solution with a concentration of from 5 to 20% by weight.

10. The process according to claim 1, which is carried out semicontinuously or entirely continuously.

11. The process according to claim 1, wherein the solvent mixture employed to synthesize the C15 salt is, optionally after restoration of the desired composition by adding at least one of the solvent components, returned to the process.

12. The process according to claim 5, wherein the solvent mixture consisting of
about 66.5% by weight methanol,
about 16.5% by weight water and
about 17% by weight heptane.

13. The process according to claim 1, wherein triphenylphosphine has a purity of about 95 to about 99.9% and the amount of triphenylphosphine employed is, based on β-vinylionol, approximately equimolar.

14. The process according to claim 1, wherein the β-vinylionol is present in about 16 to about 24% by weight and the triphenylphosphine is present about 18 to about 26% by weight.

15. The process according to claim 1, wherein the β-vinylionol is present from about 18 to about 22% by weight and the triphenylphosphine is present from about 20 to about 24% by weight.
